(19) United States
(12) Patent Application Publication
(10) Pub. No.: US 2026/0315047 A1
(43) Pub. Date: Oct. 15, 2026

(54) BIOPSY DEVICES AND METHODS OF PERFORMING A BIOPSY

(71) Applicant: BARD PERIPHERAL VASCULAR, INC., Franklin Lakes, NJ (US)

(72) Inventor: Brian C. Boyle, Chandler, AZ (US)

(73) Assignee: BARD PERIPHERAL VASCULAR, INC., Franklin Lakes, NJ (US)

(21) Appl. No.: 19/485,027

(22) PCT Filed: May 18, 2023
(86) PCT No.: PCT/US2023/022680

§ 371 Date: Nov. 17, 2025

(51) Int. Cl. A61B 10/02

(52) U.S. Cl. CPC A61B 10/0275; A61B 10/0283; A61B 2010/0208

(57) ABSTRACT

A biopsy device includes a sample notch cannula having a proximal end and a distal end, and sample notch that receives tissue from a target site. A cutting cannula is located within the sample notch cannula, and is retractable between an open position and a closed position. An outer cannula having a first end and a second end is coaxially positioned about the sample notch cannula and is retractable between a sealed position and a venting position. A vacuum assembly is fluidly coupled to the first end of the outer cannula and the proximal end of the sample notch cannula, and provides a vacuum to the sample notch cannula and the outer cannula. The outer cannula applies vacuum to a surface of the target site to seal the sample notch cannula from atmosphere in the sealed position.

### TECHNICAL FIELD

The present disclosure relates to biopsy devices, and, more particularly, to a vacuum assisted biopsy devices and methods of performing vacuum assisted biopsy.

### BACKGROUND

Traditional biopsy devices typically operate by utilizing a vacuum to pull tissue into a sample notch of the biopsy device, after which a cutting mechanism is advanced over the tissue to sever the tissue from a target site. However, vacuum loss around the biopsy device may decrease the volume of tissue that is drawn into the sample notch during a biopsy procedure, such that the acquired tissue severed from the target site may be insufficient to provide reliable diagnostic results.

### SUMMARY

An object of the present disclosure is to provide a biopsy device that is capable of acquiring large tissue samples and is further able to easily acquire tissue samples from calcified and/or other similarly diseased target sites.

In one embodiment, a biopsy device is disclosed. The biopsy device includes a sample notch cannula having a proximal end and a distal end, and sample notch that receives tissue from a target site. A cutting cannula is located within the sample notch cannula, and is retractable between an open position and a closed position. An outer cannula having a first end and a second end is coaxially positioned about the sample notch cannula and is retractable between a sealed position and a venting position. A vacuum assembly is fluidly coupled to the first end of the outer cannula and the proximal end of the sample notch cannula, and provides a vacuum to the sample notch cannula and the outer cannula. The outer cannula applies vacuum to a surface of the target site to seal the sample notch cannula from atmosphere in the sealed position.

In another embodiment, a biopsy device is disclosed. The biopsy device, includes a sample notch cannula having a proximal end, a distal end, and a plurality of ports disposed between the distal end and the proximal end, with the distal end further having a sample notch configured to receive tissue from a target site. A cutting cannula having a plurality of ports is internally located within the sample notch cannula and is retractable between an open position and a closed position. The biopsy device further includes an outer cannula having a first end and a second end, and the outer cannula is coaxially positioned about the sample notch cannula. A vacuum assembly is fluidly coupled to the first end of the outer cannula and the sample notch cannula, and provides a vacuum to the sample notch cannula and the outer cannula. The plurality of ports of the cutting cannula are aligned with the plurality of ports of the sample notch cannula when the cutting cannula is in the open position and the plurality of ports of the cutting cannula are misaligned with the plurality of ports of the sample notch cannula when the cutting cannula is in the closed position.

In yet another embodiment, a method of performing a biopsy is disclosed. The method involves inserting a biopsy device having a sample notch cannula, a cutting cannula internally located within the sample notch cannula, an outer cannula coaxially positioned about the sample notch cannula, and a vacuum assembly fluidly coupled to the outer cannula and the sample notch cannula, into a target site; advancing the outer cannula over the sample notch cannula from a venting position to a sealed position such that the outer cannula contacts an external surface of the target site; applying vacuum from the vacuum assembly to the outer cannula such that the outer cannula creates a seal between the external surface of the target site and atmosphere; applying vacuum from the vacuum assembly to the sample notch cannula to draw a tissue sample from the target site into a sample notch positioned on a distal end of the sample notch cannula; and advancing the cutting cannula from an open position to a closed position such that the cutting cannula severs the tissue sample from the target site.

These and additional features provided by the embodiments described herein will be more fully understood in view of the following detailed description, in conjunction with the drawings.

### BRIEF DESCRIPTION OF THE DRAWINGS

The embodiments set forth in the drawings are illustrative and exemplary in nature and not intended to limit the subject matter defined by the claims. The following detailed description of the illustrative embodiments can be understood when read in conjunction with the following drawings, where like structure is indicated with like reference numerals and in which:

FIG. 1 is a perspective view of an illustrative biopsy device, according to one or more embodiments described herein;

FIG. 2A is a partial cross-sectional view of the biopsy device of FIG. 1 having a cutting cannula in an open position, according to one or more embodiments described herein;

FIG. 2B is a partial cross-sectional view of the biopsy device of FIG. 2A having the cutting cannula in a closed position, according to one or more embodiments described herein;

FIG. 3A is a partial cross-sectional view of the biopsy device of FIG. 1 having an outer cannula in a venting position, according to one or more embodiments described herein;

FIG. 3B is a partial cross-sectional view of the biopsy device of FIG. 3A having the outer cannula in a sealed position, according to one or more embodiments shown and described herein;

FIG. 4A is a partial perspective view of an outer cannula of the biopsy device of FIG. 1, according to one or more embodiments shown and described herein;

FIG. 4B is a partial perspective view of the outer cannula of the biopsy device of FIG. 4A after being rotated about a sample notch cannula, according to one or more embodiments shown and described herein;

FIG. 5 depicts a schematic view of a controller for operating the biopsy device of FIG. 1, according to one or more embodiments shown and described herein; and

FIG. 6 depicts an illustrative flow diagram of a method of performing a biopsy using the biopsy device of FIG. 1, according to one or more embodiments described herein.

Corresponding reference characters indicate corresponding parts throughout the several views. The exemplifications set out herein illustrate at least one embodiment of the present disclosure, and such exemplifications are not to be construed as limiting the scope of the present disclosure in any manner.

### DETAILED DESCRIPTION

Embodiments disclosed herein relate to biopsy devices and methods of performing biopsy procedures. For example, in embodiments, a biopsy device includes a sample notch cannula, a cutting cannula internally located within the sample notch cannula, an outer cannula coaxially disposed about the sample notch cannula, and a vacuum assembly for providing a vacuum to the outer cannula and the sample notch cannula. The outer cannula is retractable about the sample notch cannula between a venting position and a sealed position such that, in the sealed position, the outer cannula seals the sample notch cannula from an external atmosphere. The sample notch cannula and the cutting cannula further includes a plurality of ports, which may be used to selectively ventilate the sample notch cannula when the plurality of ports are aligned. When a vacuum is applied to the sample notch cannula and the plurality of ports are aligned, the vacuum may cause tissue positioned at a target site to collapse around the sample notch cannula, which may further aid in sealing the sample notch cannula from the external atmosphere. By sealing the sample notch cannula, vacuum loss along the sample notch cannula is minimized, which allows for a maximum vacuum force to be generated within a sample notch of the sample notch cannula. By increasing the vacuum operating within the sample notch, the biopsy device is able to more easily and efficiently acquire larger tissue samples from a target site, which allows for increased diagnostic results when testing the tissue sample and alleviation of the need to acquire multiple tissue samples from the target site and/or perform multiple biopsy procedures.

Embodiments of biopsy devices and methods of performing biopsy procedures will now be described in more detail herein with reference to the drawings and where like numbers refer to like structures.

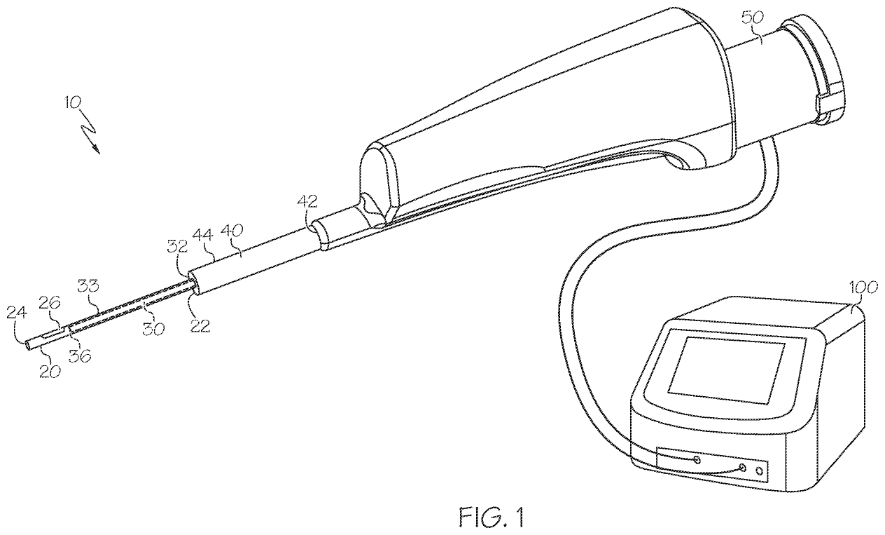

Referring now to FIG. 1, an illustrative biopsy device 10 is depicted. The biopsy device 10 includes a sample notch cannula 20, a cutting cannula 30, an outer cannula 40, and a vacuum assembly 50. In these embodiments, the sample notch cannula 20 includes a proximal portion 22, a distal portion 24 opposite the proximal portion 22, and a sample notch 26 positioned along the distal portion 24 of the sample notch cannula 20. As will be described in additional detail herein, the sample notch 26 is operable to receive tissue located at a target site during a biopsy procedure.

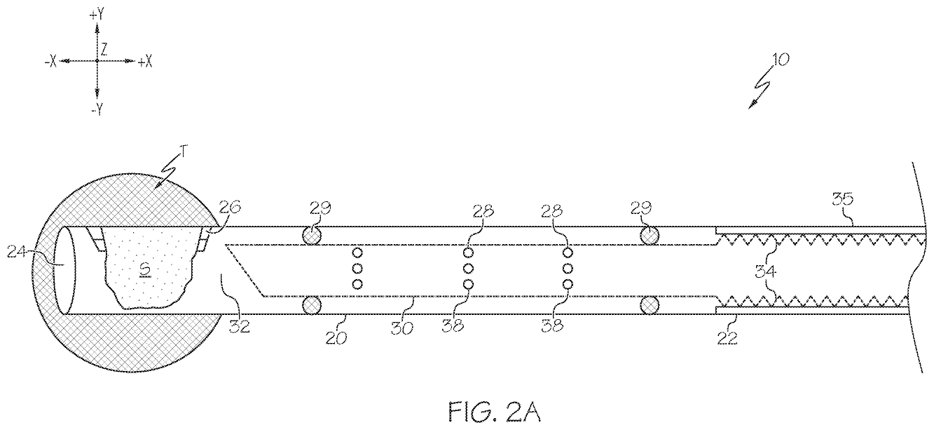

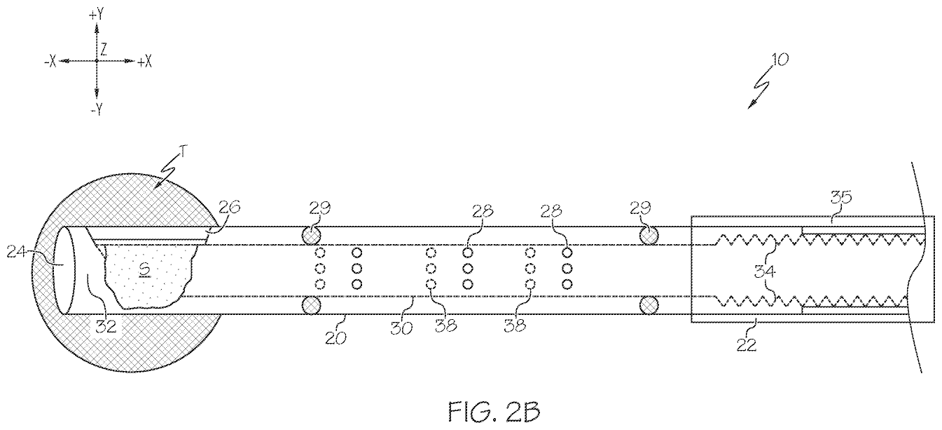

Referring still to FIG. 1, the cutting cannula 30 is internally located within the sample notch cannula 20. In these embodiments, the cutting cannula 30 includes a first end 32 and a second end 33, with the second end 33 including a cutting edge 36. For example, the cutting edge 36 may include a serrated edge, a tapered edge, or any other similar edge capable of severing tissue from a target site. As will be described in additional detail herein with reference to FIGS. 2A and 2B the cutting cannula 30 is translatable between an open position and a closed position, such that, when the cutting cannula 30 is translated from the open position to the closed position, the cutting edge 36 of the cutting cannula 30 advances through the sample notch 26 of the sample notch cannula 20 and severs tissue drawn into the sample notch 26 from the target site.

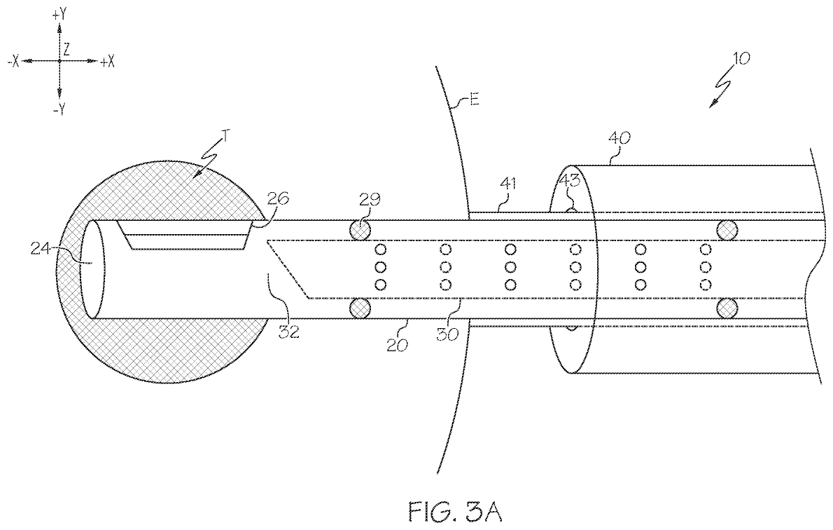

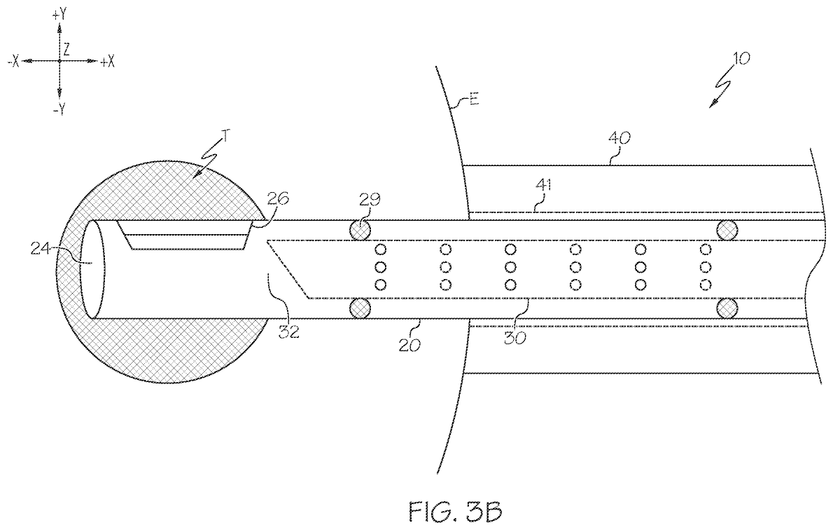

Referring still to FIG. 1, the outer cannula 40 further includes a first end 42 and a second end 44, and is coaxially positioned about the sample notch cannula 20. In these embodiments, the outer cannula 40 is retractable between a sealed position and a venting position, as will be described in additional detail herein with reference to FIGS. 3A and 3B.

In the embodiments described herein, the sample notch cannula 20 and the cutting cannula 30 may be formed of stainless steel, titanium, or any other similarly biocompatible metal. As defined herein, biocompatible materials may refer to materials suitable for use in medical devices, including materials with medical grade strength, durability, and corrosion resistance.

As further depicted in FIG. 1, the vacuum assembly 50 is fluidly coupled to the first end 42 of the outer cannula 40 and the proximal portion 22 of the sample notch cannula 20. Accordingly, the vacuum assembly 50 generates negative pressure within both the outer cannula 40 and the sample notch cannula 20. For example, negative pressure generated within the sample notch cannula 20 creates a pressure differential between the sample notch cannula 20 and the target site in which the biopsy device 10 is inserted. The negative pressure within the sample notch cannula 20 causes tissue positioned at the target site to be drawn into the sample notch 26, at which point the tissue may be captured for analysis. Similarly, negative pressure generated within the outer cannula 40 creates a pressure differential between the outer cannula 40 and an external environment (e.g., atmosphere). Accordingly, the negative pressure within the outer cannula 40 acts to seal the outer cannula 40 to an external surface of the target site in which the biopsy device 10 is deployed, such that the biopsy device is sealed from the external environment. In the embodiments described herein, the vacuum assembly 50 further includes a pump, such as a vacuum pump, or any other similar mechanism capable of generating negative pressure within the biopsy device 10.

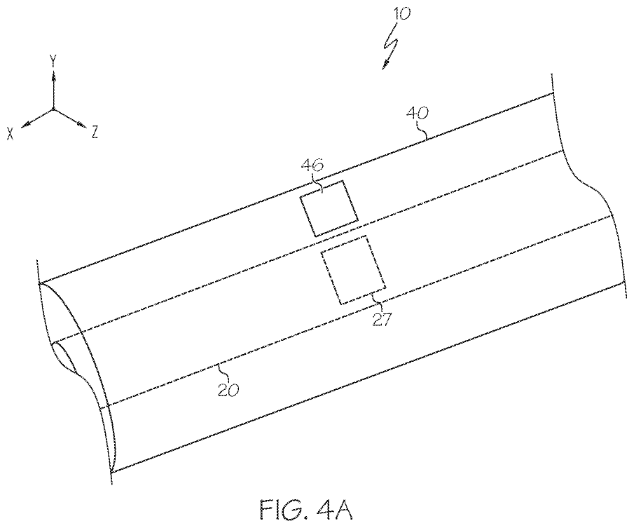

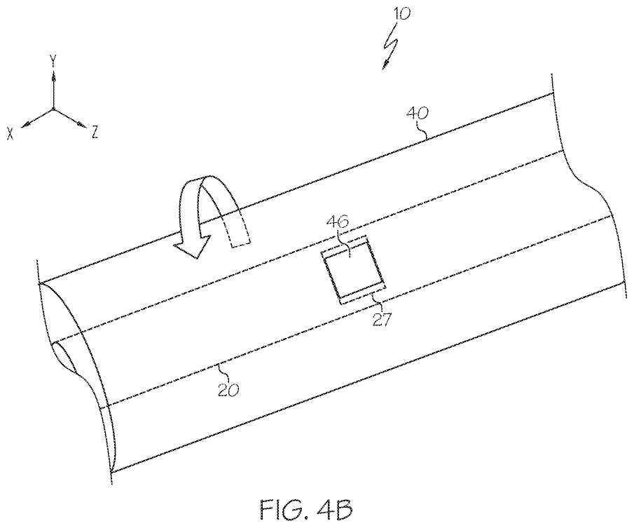

Referring still to FIG. 1, the biopsy device 10 further includes a controller 100 that controls operation of the biopsy device 10. For example, in some embodiments, the controller 100 may be operated to control movement of the cutting cannula 30 between the open position and the closed position, movement of the outer cannula 40 between the sealed position and the venting position, activation of the vacuum assembly 50, and other similar functions of the biopsy device 10. Although not depicted, it should be further noted that in some embodiments, the controller 100 may be integrated into the biopsy device 10. For example, the biopsy device 10 may include a handle having a plurality of control buttons for providing user control over the various functions of the biopsy device 10 described herein. The operation of the controller 100 of the biopsy device 10 will be described in additional detail herein with reference to FIG. 4.

Turning now to FIGS. 2A and 2B, a partial cross-sectional view of the biopsy device 10 is illustrated with the cutting cannula 30 depicted in the open position and the closed position, respectively. In these embodiments, the cutting cannula 30 further includes a plurality of ports 38, and the sample notch cannula 20 similarly includes a plurality of ports 28. The plurality of ports 28 of the sample notch cannula 20 may extend along a length of the sample notch cannula 20 (e.g., between the distal portion 24 of the sample notch cannula 20 and the proximal portion 22 of the sample notch cannula 20) and may include any number of ports. For example, although the sample notch cannula 20 is depicted as including three ports 28, the sample notch cannula 20 may include two ports, four ports, a single port, or any other number of ports without departing from the scope of the present disclosure.

Referring still to FIGS. 2A and 2B, the plurality of ports 38 of the cutting cannula 30 may similarly extend along a length of the cutting cannula 30. In these embodiments, the plurality of ports 38 of the cutting cannula 30 may include the same number of ports as the plurality of ports 28 of the sample notch cannula 20. By providing the same number of ports on both the sample notch cannula 20 and the cutting cannula 30, the plurality of ports 28 of the sample notch cannula 20 and the plurality of ports 38 of the cutting cannula 30 may be used to provide ventilation to the sample notch cannula 20 and the cutting cannula 30 as the cutting cannula 30 is translated between the open position and the closed position.

As noted herein, the cutting cannula 30 may be translatable between the open position (FIG. 2A) and the closed position (FIG. 2B), such that the cutting cannula 30 is capable of moving in a linear direction (e.g., in the +/−x-direction as depicted in the coordinate axes of FIGS. 2A-2B) through the sample notch cannula 20. In these embodiments, the cutting cannula 30 may include a translation mechanism, which may be operated to move the cutting cannula 30 forward (e.g., in the −x direction as depicted in the coordinate axes of FIGS. 2A-2B) from the open position to the closed position, and backwards (e.g., in the +x direction as depicted in the coordinate axes of FIGS. 2A-2B) from the closed position to the open position through the sample notch cannula 20.

For example, in these embodiments, the cutting cannula 30 may further include a cutting spindle 34 positioned on a first end 32 of the cutting cannula 30. To position the cutting cannula 30 in the open position, the cutting spindle 34 of the cutting cannula 30 may be rotated in a first direction (e.g., clockwise) such that the cutting spindle 34 is driven into a cutting nut 35 positioned within the sample notch cannula 20 (e.g., in the +x-direction as depicted in the coordinate axes of FIGS. 2A-2B). As the cutting spindle 34 is threaded into the cutting nut 35, the cutting cannula 30 may be linearly translated towards the proximal portion 22 of the sample notch cannula 20 (e.g., in the +x direction as depicted in the coordinate axes of FIGS. 2A-2B), such that the cutting cannula 30 is moved to the open position.

To move the cutting cannula 30 to the closed position, the cutting spindle 34 may be rotated in a second direction opposite the first direction (e.g., counterclockwise), such that the cutting spindle 34 is driven out of the cutting nut 35. As the cutting spindle 34 is driven out of the cutting nut 35, the cutting cannula 30 may advance in a longitudinal direction towards the distal portion 24 of the sample notch cannula 20 (e.g., in the −x-direction depicted in the coordinate axes of FIGS. 2A-2B, such that the cutting edge 36 of the cutting cannula 30 advances past a tissue sample positioned within the sample notch 26 and severs the tissue sample from the target site. Thus, cutting cannula 30 may have a rotational cutting motion and may be translated axially. In these embodiments, the pitch of the threads of cutting spindle 34 may determine the number of revolutions per axial distance that cutting cannula 230 moves axially. It should be appreciated that, in the embodiments described herein, the threads of the cutting spindle 34 may be sized to ensure that the cutting edge 36 is adjacent the sample notch 26 when the cutting cannula 30 advances past the sample notch 26.

Although the translation mechanism is depicted as including the cutting spindle 34 and cutting nut 35 in FIGS. 2A-2B, it should be appreciated that the translation mechanism may be any mechanism capable of driving the cutting cannula 30 in the linear direction between the open position and the closed position. For example, the translation mechanism may include a sliding mechanism including a set of rails or grooves on the sample notch cannula 20 that guide the cutting cannula 30 as the cutting cannula 30 translates between the open position and the closed position. In these embodiments, rotation of the threaded rod or screw may be translated into linear motion, such that the cutting cannula 30 may translate between the open position and the closed position. In other embodiments, the translation mechanism may include a spring loaded mechanism, a hydraulic or pneumatic mechanism, or any other similar mechanism capable of translating the cutting cannula 30 between the open position and the closed position, as has been described herein.

As further depicted in FIGS. 2A and 2B, the sample notch cannula 20 may include a plurality of o-rings 29 that may provide a seal between the cutting cannula 30 and the sample notch cannula 20. In these embodiments, the plurality of o-rings 29 may be disposed about the plurality of ports 28 positioned on the sample notch cannula 20, such that the plurality of o-rings 29 may prevent tissue and/or debris from entering the space between the sample notch cannula 20 and the cutting cannula 30. The plurality of o-rings 29 may be formed of a flexible, durable material such as silicone or rubber, such that the plurality of o-rings 29 allow for a seal to be formed between the cutting cannula 30 and the sample notch cannula 20 while still allowing for the cutting cannula 30 to translate freely (e.g., between the open position and the closed position) within the sample notch cannula 20.

Referring now to FIG. 2A, the cutting cannula 30 is depicted within the sample notch cannula 20 in the open position. As illustrated in FIG. 2A, when the cutting cannula 30 is in the open position, the plurality of ports 38 of the cutting cannula 30 align with the plurality of ports 28 of the sample notch cannula 20. Accordingly, when the vacuum assembly is activated, the negative pressure generated within the sample notch cannula 20 and the cutting cannula 30 may cause tissue positioned within a target site T to collapse around the sample notch cannula 20 such that the sample notch cannula 20 and cutting cannula 30 are sealed from an external environment.

In these embodiments, it may be possible to minimize vacuum loss along the sample notch cannula 20 between the target site T and an external environment by collapsing tissue within the target site T around the sample notch cannula 20. For example, when tissue collapses against the plurality of ports 28 of the sample notch cannula 20, contact between the sample notch cannula 20 and the collapsed tissue may act to seal the sample notch cannula 20 from an external environment. The seal formed between the collapsed tissue and the sample notch cannula 20 may help prevent the negative pressure generated within the sample notch cannula 20 from escaping the target site T.

As tissue at the target site T collapses around the sample notch cannula 20, it should be understood that tissue may be drawn into the sample notch 26, as is depicted in FIG. 2A. By minimizing vacuum loss along the sample notch cannula 20, it may be possible to generate and maintain a more substantial negative pressure at the sample notch 26, which may allow for a greater volume of tissue to be drawn into the sample notch 26 than what is achievable utilizing a sample notch cannula that does not include the plurality of ports 28. By increasing the volume of tissue drawn into the sample notch 26 of the sample notch cannula 20, it may be possible to obtain larger tissue sample S during a biopsy procedure, which may allow for better diagnostic results while minimizing and/or eliminating the need to acquire multiple tissue samples from multiple target site Ts.

Referring now to FIG. 2B, the cutting cannula 30 is depicted in the closed position. In these embodiments, once the tissue sample S has been drawn into the sample notch 26 of the sample notch cannula 20, the cutting cannula 30 may be actuated such that the cutting cannula 30 moves in a longitudinal direction from the open position to the closed position, as has been described in detail herein.

As the cutting cannula 30 advances (e.g., in the −x direction as depicted in the coordinate axis of FIG. 2B), the plurality of ports 38 of the cutting cannula 30 become misaligned with the plurality of ports 28 of the sample notch cannula 20. Furthermore, the cutting cannula 30 traverses the tissue sample S that has been drawn into the sample notch 26, such that the cutting edge 36 of the cutting cannula 30 passes through the tissue sample S drawn into the target site T and severs the tissue sample S from the target site T.

Referring still to FIG. 2B, the misalignment of the plurality of ports 28 of the sample notch cannula 20 and the plurality of ports 38 of the cutting cannula 30 act to seal the tissue sample S within the biopsy device 10. For example, once the plurality of ports 28, 38 are misaligned, the negative pressure generated by the vacuum assembly may create a pressure differential between the sample notch cannula 20 and the target site T. In these embodiments, the pressure differential may cause the tissue sample S to become sealed in the cutting cannula 30. With the tissue sample S sealed within the cutting cannula 30, the negative pressure within the sample notch cannula 20 and the cutting cannula 30 may draw the tissue sample through the cutting cannula 30 (e.g., in the +x-direction as depicted in the coordinate axis of FIG. 2B) and deposited into the vacuum assembly.

Referring still to FIG. 2B, the vacuum assembly may further include a sample basket or other similar structure capable of storing the tissue sample S acquired from a target site T during a biopsy procedure. In other embodiments, the tissue sample S may remain confined in the sample notch 26 of the sample notch cannula 20 after the cutting cannula 30 is advanced to the closed position, and may be removed when the biopsy device 10 is removed from the target site T (e.g., at the conclusion of a biopsy procedure or the like).

Turning now to FIGS. 3A and 3B, the outer cannula 40 of the biopsy device 10 is depicted in the venting position and the sealed position, respectively. In these embodiments, the outer cannula 40 may be capable of moving in a linear direction (e.g., the +/−x-direction as depicted in the coordinate axes of FIGS. 3A-3B) along the sample notch cannula 20. In these embodiments, the outer cannula 40 may include an actuation mechanism, which may be operated to move the outer cannula forward (e.g., in the −x direction as depicted in the coordinate axes of FIGS. 3A-3B) from the venting position to the sealed position, and backwards (e.g., in the +x direction as depicted in the coordinate axes of FIGS. 3A-3B) from the sealed position to the venting position, as will be described in additional detail herein.

For example, as depicted in FIGS. 3A-3B, the sample notch cannula 20 may include a plurality of rails 41 spaced in a circumferential direction about the sample notch cannula 20 and extending from the proximal portion 22 to the distal portion 24 of the sample notch cannula 20 in a longitudinal direction (e.g., in the +/−x-direction as depicted in the coordinate axes of FIGS. 3A-3B. In these embodiments, the outer cannula 40 may include a plurality of slots 43 which correspond to the plurality of rails 41 of the sample notch cannula 20. For example, the plurality of slots 43 of the outer cannula 40 may be similarly disposed in a circumferential direction about an inner surface of the outer cannula 40 and extend from the first end 42 to the second end 44 of the outer cannula 40 in the longitudinal direction. In these embodiments, the plurality of slots 43 of the outer cannula 40 may engage the plurality of rails 41 of the sample notch cannula 20, such that the outer cannula 40 may translate along the plurality of rails 41 of the sample notch cannula 20 as the outer cannula 40 translates from the sealed position to the venting position.

In these embodiments, the engagement between the plurality of rails 41 of the sample notch cannula 20 and the plurality of slots 43 of the outer cannula 40 may aid in guiding the outer cannula 20 in the longitudinal direction (e.g., in the +/−x-direction as depicted in the coordinate axes of FIGS. 3A-3B). For example, the plurality of rails 41 may ensure that the outer cannula 40 remains in alignment with the external surface O of the target site T as the outer cannula 40 translates. By maintaining alignment of the outer cannula 40 and the external surface O, a user may ensure that the outer cannula 40 is effectively sealed to the external surface O of the target site when the outer cannula 40 is advanced to the sealed position.

Although the actuation mechanism is depicted as including a plurality of slots and rails, it should be appreciated that the actuation mechanism may be any mechanism capable of driving the outer cannula 40 in the linear direction between the sealed position and the venting position. For example, the actuation mechanism may include a screw mechanism including a threaded rod or screw attached to the outer cannula 40. In these embodiments, rotation of the threaded rod or screw may be translated into linear motion, such that the outer cannula 40 may translate between the sealed position and the venting position. In other embodiments, the translation mechanism may include a spring loaded mechanism, a hydraulic or pneumatic mechanism, or any other similar mechanism capable of translating the outer cannula 40 between the open position and the closed position, as has been described herein.

Referring now to FIG. 3A, the outer cannula 40 is depicted in the venting position. In this embodiment, the outer cannula 40 may be positioned away from an external surface E of the target site T, such that target site T is exposed to an external environment (e.g., atmosphere) and may be ventilated. For example, as depicted in FIG. 3A, negative pressure generated within the biopsy device 10 may escape the target site T via external surface E, which may allow for various levels of ventilation within the target site T.

In the embodiments described herein, the outer cannula 40 may be translated to the venting position while the biopsy device 10 is being positioned within the target site T. Once the biopsy device 10 is positioned within the target site T, the outer cannula 40 may be advanced to the sealed position, as is depicted in FIG. 3B.

In the sealed position, the outer cannula 40 contacts the external surface E of the target site T. In some embodiments, the outer cannula 40 may include a sealing mechanism, such as an o-ring or other similar component, which may allow for a seal to be formed between the outer cannula 40 and the external surface E of the target site. In these embodiments, the outer cannula 40 may be advanced (e.g., in the −x direction as depicted in the coordinate axis of FIG. 3B) until the sealing mechanism is secured to the external surface E of the target site T.

Referring still to FIG. 3B, with the outer cannula 40 secured to the external surface E of the target site T, the vacuum assembly may generate negative pressure within the outer cannula 40, such that the outer cannula 40 may be sealed to the external surface E of the target site T. In these embodiments, the seal formed between the outer cannula 40 and the external surface E of the target site T may effectively seal the biopsy device 10 from an external environment (e.g., atmosphere). Accordingly, by sealing the outer cannula 40 to the external surface E of the target site T, a user may ensure that negative pressure generated within the sample notch cannula 20 does not escape the target site T, such that vacuum loss within the target site T is minimized and/or eliminated.

In addition to minimizing vacuum loss within the target site T, the outer cannula 40 may further aid in minimizing blood loss from the external surface E of the target site T when the outer cannula 40 is in the sealed position. For example, in some embodiments, the outer cannula 40 may further include a fluid line which extends from the outer cannula 40 to the vacuum assembly. In these embodiments, the negative pressure generated within the outer cannula 40 may cause blood and/or other fluids to be drawn from the external surface E of the target site T and into the fluid line, which may then transport the blood and/or other fluid away from the target site T. Furthermore, the pressure applied to the external surface E of the target site T may reduce blood flow to the target site T, such that the amount of blood and/or other fluids produced during the biopsy procedure are similarly minimized.

In these embodiments, negative pressure may be continuously applied to the outer cannula 40 to ensure that the outer cannula 40 remains sealed to the external surface E of the target site T throughout the entirety of a biopsy procedure. Once the biopsy procedure has been completed, the vacuum assembly may be deactivated, such that the pressure within the outer cannula 40 is alleviated and the outer cannula 40 may be retracted to the venting position.

Turning now to FIGS. 4A and 4B, in some embodiments, the outer cannula 40 may further include a ventilation aperture 46, which may be used to selectively ventilate the target site T without translating the outer cannula 40 from the sealed position to the venting position. For example, the target site T may be selectively ventilated by aligning the ventilation aperture 46 of the outer cannula 40 with a ventilation aperture 27 positioned on the sample notch cannula 20.

For example, the outer cannula 40 may be rotatable about the sample notch cannula 20 (e.g., rotated about the x-axis as depicted in FIGS. 4A and 4B). Accordingly, the outer cannula 40 may be rotated to selectively control ventilation of the target site T. For example, as depicted in FIG. 4A, when the ventilation aperture 46 of the outer cannula 40 and the ventilation aperture 27 of the sample notch cannula 20 are misaligned, the target site T may remain sealed from the atmosphere outside the external surface E of the target site T. It should be appreciated that, in these embodiments, the sealing mechanism disposed on the outer cannula 40 may aid in maintaining the seal between external surface E of the target site T and the outer cannula 40, as has been described herein with reference to FIGS. 3A-3B. However, by rotating the outer cannula 40 about the sample notch cannula 20, the ventilation aperture 46 of the outer cannula 40 and the ventilation aperture 27 of the sample notch cannula 20 may be moved into alignment, as illustrated in FIG. 4B (or partial alignment, not shown), such that the target site T is exposed to atmosphere via the ventilation apertures 27, 46.

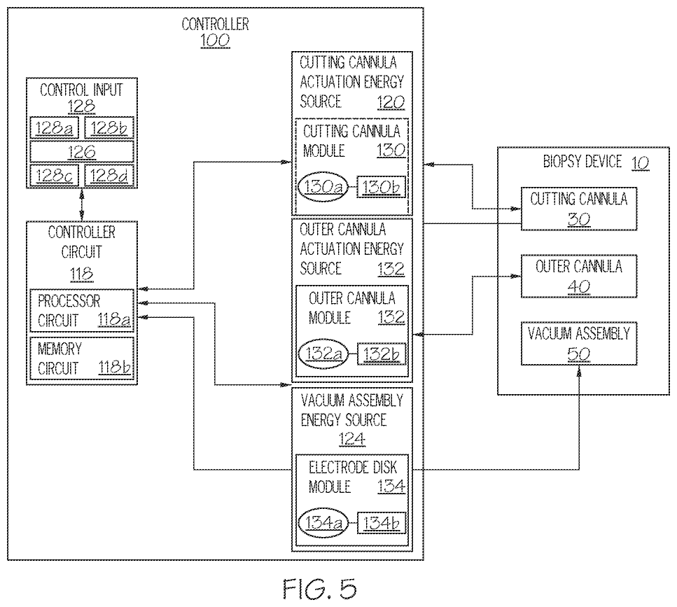

Referring now to FIG. 5, a schematic view of the controller 100 for operating the biopsy device 10 is illustrated. In some embodiments, the controller 100 may be configured to actuate the cutting cannula 30 between the open position and the closed position, actuate the outer cannula 40 between the sealed position and the venting position, and control activation of the vacuum assembly 50. The controller 100 may include a controller circuit 118, a cutting cannula actuation energy source 120, an outer cannula actuation energy source 122, and a vacuum assembly energy source 124. The controller 100 may further include a control input 128, such as a computer, which may allow a user to operate the controller 100. In these embodiments, the control input 128 may be configured to provide control of the controller 100 via a software program, or may be manually controlled via a user manipulating a user interface thereof.

Referring still to FIG. 5, the control input 128 may include control buttons and visual/aural indicators, such as a display and/or speakers, with the control buttons providing user control over various functions of the controller 100, and with the visual/aural indicators providing visual/aural feedback of the status of one or more conditions and/or positions of components of the controller 100. For example, the display and/or speakers may provide an audible tone that indicates to a user that the biopsy device 10 is positioned within the target area, that the cutting cannula 30 has been moved to the open or closed position, that the outer cannula has been moved to the sealed or venting position, and/or that the vacuum assembly 50 has been activated. The control buttons may include one or more buttons 128a, 128b for actuating the cutting cannula 30 between the open position and the closed position, actuating the outer cannula 40 between the sealed position and the venting position, and one or more buttons and/or knobs for adjusting the vacuum (e.g., negative pressure) generated within the biopsy device 10 by the vacuum assembly 50.

Controller circuit 118 is electrically and communicatively coupled to cutting cannula actuation energy source 120, outer cannula actuation energy source 122, vacuum assembly energy source 124 and control input 128, such as by one or more wires or circuit traces. Controller circuit 118 may be assembled on an electrical circuit and may include, for example, a processor circuit 118a and a memory circuit 118b.

Processor circuit 118a has one or more programmable microprocessors and associated circuitry, such as an input/output interface, buffers, memory, etc. Memory circuit 118b is communicatively coupled to processor circuit 118a, e.g., via a bus circuit, and is a non-transitory electronic memory that may include volatile memory circuits, such as random access memory (RAM), and non-volatile memory circuits, such as read only memory (ROM), electronically erasable programmable ROM (EEPROM), flash memory, etc. Controller circuit 118 may be formed as one or more Application Specific Integrated Circuits (ASIC).

Controller circuit 118 is configured via software and/or firmware residing in memory circuit 118b to execute program instructions to perform functions associated with the actuation of the cutting cannula 30, outer cannula 40, and/or vacuum generated within the biopsy device by the vacuum assembly 50.

Referring still to FIG. 5, cutting cannula actuation energy source 120 may include, for example, a cutting cannula module 130. In these embodiments, the cutting cannula actuation energy source 120 may include a cutting cannula module 130 that may be electrically and controllably coupled to controller circuit 118. As provided herein, the cutting cannula module 130 may be electrically coupled to the controller circuit 118 by way of electrical wiring or any other suitable electrical connections, such that user inputs on the control input 128 may be relayed to the controller circuit 118 and used to control the power delivered by the cutting cannula module 130 to the cutting cannula 30. In these embodiments, the cutting cannula module 130 may include a power supply 130a, such as an electric motor, to which an electric lead 130b is attached.

Referring still to FIG. 5, outer cannula actuation energy source 122 may include, for example, an outer cannula module 132. In these embodiments, the outer cannula actuation energy source 122 may include an outer cannula module 132 that may be electrically and controllably coupled to controller circuit 118. As provided herein, the outer cannula module 132 may be electrically coupled to the controller circuit 118 by way of electrical wiring or any other suitable electrical connections, such that user inputs on the control input 128 may be relayed to the controller circuit 118 and used to control the power delivered by the outer cannula module 132 to the outer cannula 40. In these embodiments, the outer cannula module 132 may include a power supply 132a, such as an electric motor, to which an electric lead 130b is attached.

As further depicted in FIG. 5, the vacuum assembly energy source 124 may include may include a vacuum assembly module 134 to control the vacuum generated within the biopsy device 10 by the vacuum assembly 50. For example, as depicted in FIG. 4, the vacuum assembly energy source 124 may include a power supply 134a, such as an electric motor, that activates the vacuum assembly 50 via an electric lead 134b. In these embodiments, the user may adjust the vacuum generated within the biopsy device 10 by altering the power supplied to the vacuum assembly 50 via the vacuum assembly module 134.

Referring still to FIG. 5, in the embodiments described herein, the controller 100 may be electrically and operably connected to the proximal portion 22 of the sample notch cannula 20 and the first end 42 of the outer cannula 40 via the vacuum assembly 50. The electric leads 130b, 132b, 134b may extend from the controller 100 through the vacuum assembly 50 and into the proximal portion 22 of the sample notch cannula 20 and the first end 42 of the outer cannula 40, such that the cutting cannula actuation energy source 120 and the outer cannula actuation energy source 122 are able to control the actuation of the cutting cannula 30 and the outer cannula 40, respectively. For example, once the biopsy device 10 is positioned within the target site, the outer cannula actuation energy source 122 may be operated to cause the outer cannula 40 to move from the venting position to the sealed position, such that the outer cannula 40 contacts an external surface of the target site and seals the biopsy device 10 from atmosphere. As described herein, with the outer cannula 40 positioned adjacent the external surface of the target site, the vacuum assembly 50 may be activated, such that negative pressure is generated within the outer cannula 40. In these embodiments, the negative pressure formed within the outer cannula 40 may act to seal outer cannula 40 to an external surface of the target site.

With the outer cannula 40 sealed against the external surface of the target site, the cutting cannula actuation energy source 120 may be activated to translate the cutting cannula 30 from the open position to the closed position, such that the cutting cannula 30 severs a tissue sample drawn into the sample notch of the sample notch cannula, as has been described in detail herein. Once the cutting cannula 30 has severed tissue from the target site, the tissue may be transported through the cutting cannula 30 and into the vacuum assembly 50, at which point the tissue may be removed from the biopsy device 10 and acquired for diagnostic testing. It should be understood that, in the embodiments described herein, the vacuum assembly 50 may remain active throughout the entirety of a biopsy procedure. Furthermore, the vacuum assembly 50 may be adjusted (e.g., manually or automatically via the controller 100) throughout the biopsy procedure to ensure that sufficient vacuum is generated and maintained within the outer cannula 40 and the sample notch cannula 20.

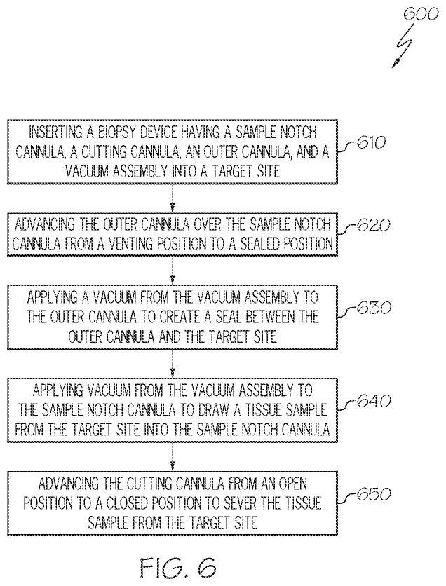

Turning now to FIG. 6, an illustrative method 600 of performing a biopsy utilizing the biopsy device depicted in FIGS. 1-5B is disclosed. In embodiments, the method 600 includes inserting the biopsy device 10 into a target site T, as is depicted at block 610. In these embodiments, the outer cannula 40 may be translated to the venting position and the cutting cannula 30 may be positioned in the open position as the biopsy device 10 is positioned within the target site T.

With the biopsy device 10 positioned in the target site T, the method 600 advances to block 620, which includes advancing the outer cannula 40 of the biopsy device 10 over the sample notch cannula 20 from the venting position to the sealed position such that the outer cannula 40 contacts the external surface E of the target site T. As described herein, the outer cannula 40 may further include a sealing mechanism, such as an o-ring or other similar mechanism, which may allow for the outer cannula 40 to conform to the external surface E of the target site T.

Referring still to FIG. 6, the method 600 further includes applying a vacuum from the vacuum assembly 50 to the outer cannula 40, as depicted at block 630. For example, once the outer cannula 40 has contacted the external surface E of the target site T, the vacuum assembly 50 generates negative pressure within the outer cannula 40, which seals the outer cannula 40 to the external surface E of the target site T. In these embodiments, the seal formed between the outer cannula 40 and the external surface E of the target site T seals the target site T from an external environment (e.g., atmosphere).

With the outer cannula 40 sealed to the external surface E of the target site T, the method further includes applying vacuum from the vacuum assembly 50 to the sample notch cannula 20, such that a tissue sample S from the target site T is drawn into the sample notch 26, as is depicted at block 640. In these embodiments, when vacuum is applied to the sample notch cannula 20, the cutting cannula 30 is in the open position. As a result, the plurality of ports 28 located on the sample notch cannula 20 and the plurality of ports 38 positioned on the cutting cannula 30 are aligned, which may expose tissue at the target site to the vacuum generated by the vacuum assembly 50. In these embodiments, the vacuum acting on the tissue within the target site T causes the tissue to collapse around the sample notch cannula 20, which may further aid to seal the target site T from atmosphere and minimize vacuum loss along the sample notch cannula 20.

Referring still to FIG. 6, once the tissue sample S has been drawn into the sample notch 26, the method moves to block 650, which includes advancing the cutting cannula 30 from the open position to the closed position. As the cutting cannula 30 advances, the cutting cannula 30 severs the tissue sample S from the target site T, such that the tissue sample S may be removed from the target site T.

Furthermore, as the cutting cannula 30 advances, the plurality of ports 38 of the cutting cannula 30 and the plurality of ports 28 of the sample notch cannula 20 become misaligned, such that the vacuum generated by the vacuum assembly is contained within the biopsy device 10. In these embodiments, the vacuum generated by the vacuum assembly 50 acts to secure the tissue sample S within the sample notch cannula 20, as has been described herein. With the tissue sample S captured, the vacuum assembly 50 is deactivated, such that the pressure generated within the biopsy device 10 and target site T is alleviated. With the pressure alleviated, the outer cannula 40 is retracted to the venting position, and the biopsy device 10 may be removed from the target site T.

As should be appreciated in view of the foregoing, a biopsy device is described herein. The biopsy device may include a sample notch cannula, a cutting cannula internally located within the sample notch cannula, an outer cannula coaxially positioned about the sample notch cannula and retractable between a sealed position and a venting position, and a vacuum assembly that provides a vacuum to the outer cannula and the sample notch cannula. The sample notch cannula and the cutting cannula further include a plurality of ports, such that, when the plurality of ports are aligned, the vacuum generated by the vacuum assembly causes tissue within a target site to collapse around the sample notch cannula, thereby sealing the target site from atmosphere. Similarly, the outer cannula may contact an external surface of the target site in the sealed position, which may further act to seal the sample notch cannula and target site from atmosphere. By sealing the target site, the biopsy device disclosed herein may minimize vacuum loss along the sample notch cannula, which may in turn allow for larger tissue samples to be obtained during a biopsy procedure.

Embodiments may be further described with references to the following numbered clauses:

Clause 1. A biopsy device, comprising: a sample notch cannula having a proximal end and a distal end, the distal end having a sample notch configured to receive tissue from a target site; a cutting cannula being internally located within the sample notch cannula, the cutting cannula being retractable between an open position and a closed position; an outer cannula having a first end and a second end, the outer cannula being coaxially positioned about the sample notch cannula and retractable between a sealed position and a venting position; and a vacuum assembly fluidly coupled to the first end of the outer cannula and the proximal end of the sample notch cannula, the vacuum assembly configured to provide a vacuum to the sample notch cannula and the outer cannula; wherein the outer cannula applies vacuum to a surface of the target site to seal the sample notch cannula from atmosphere in the sealed position.

Clause 2. The biopsy device of clause 1, wherein the sample notch cannula comprises a plurality of ports.

Clause 3. The biopsy device of clause 1 or 2, wherein the sample notch cannula further comprises a plurality of seals.

Clause 4. The biopsy device of any of clauses 1-3, wherein the cutting cannula comprises a plurality of ports.

Clause 5. The biopsy device of any of clauses 1-4, wherein the plurality of ports of the cutting cannula align with the plurality of ports of the sample notch cannula when the cutting cannula is in the open position.

Clause 6. The biopsy device of any of clauses 1-5, wherein the plurality of ports of the cutting cannula are misaligned with the plurality of ports of the sample notch cannula when the cutting cannula is in the closed position.

Clause 7. The biopsy device of any of clauses 1-6, wherein the outer cannula disengages the surface of the target site to ventilate the sample notch cannula in the venting position.

Clause 8. The biopsy device of any of clauses 1-7, wherein the outer cannula and the sample notch cannula each include a ventilation aperture, and the outer cannula is rotatable about the sample notch cannula to selectively ventilate the sample notch cannula.

Clause 9. The biopsy device of any of clauses 1-8, wherein the sample notch cannula is sealed from the atmosphere when the ventilation aperture of the outer cannula and the ventilation aperture of the sample notch cannula are aligned.

Clause 10. The biopsy device of any of clauses 1-9, wherein the sample notch cannula is ventilated when the ventilation aperture of the outer cannula and the ventilation aperture of the sample notch cannula are misaligned.

Clause 11. A biopsy device, comprising: a sample notch cannula having a proximal end, a distal end, and a plurality of ports disposed between the distal end and the proximal end, the distal end further having a sample notch configured to receive tissue from a target site; a cutting cannula being internally located within the sample notch cannula, the cutting cannula having a plurality of ports and being retractable between an open position and a closed position; an outer cannula having a first end and a second end, the outer cannula being coaxially positioned about the sample notch; and a vacuum assembly fluidly coupled to the first end of the outer cannula and the sample notch cannula, the vacuum assembly configured to provide a vacuum to the sample notch cannula and the outer cannula; wherein the plurality of ports of the cutting cannula are aligned with the plurality of ports of the sample notch cannula when the cutting cannula is in the open position and the plurality of ports of the cutting cannula are misaligned with the plurality of ports of the sample notch cannula when the cutting cannula is in the closed position.

Clause 12. The biopsy device of clause 11, wherein the plurality of ports of the cutting cannula and the plurality of ports in the sample notch cannula allow the vacuum assembly to apply a vacuum to the sample notch cannula when aligned.

Clause 13. The biopsy device of clause 11 or 12, wherein the plurality of ports of the cutting cannula and the plurality of ports in the sample notch cannula seal the sample notch cannula from atmosphere when misaligned.

Clause 14. The biopsy device of any of clauses 11-13, wherein the outer cannula is further retractable between a sealed position and a venting position.

Clause 15. The biopsy device of any of clauses 11-14, wherein the outer cannula applies vacuum to a surface of the target site to seal the sample notch cannula from atmosphere in the sealed position.

Clause 16. The biopsy device of any of clauses 11-15, wherein the outer cannula disengages the surface of the target site to ventilate the sample notch cannula in the venting position.

Clause 17. A method of performing a biopsy comprising: inserting a biopsy device having a sample notch cannula, a cutting cannula internally located within the sample notch cannula, an outer cannula coaxially positioned about the sample notch cannula, and a vacuum assembly fluidly coupled to the outer cannula and the sample notch cannula, into a target site; advancing the outer cannula over the sample notch cannula from a venting position to a sealed position such that the outer cannula contacts an external surface of the target site; applying vacuum from the vacuum assembly to the outer cannula such that the outer cannula creates a seal between the external surface of the target site and atmosphere; applying vacuum from the vacuum assembly to the sample notch cannula to draw a tissue sample from the target site into a sample notch positioned on a distal end of the sample notch cannula; and advancing the cutting cannula from an open position to a closed position such that the cutting cannula severs the tissue sample from the target site.

Clause 18. The method of clause 17, wherein the cutting cannula includes include a plurality of ports.

Clause 19. The method of clause 17 or 18, wherein the sample notch cannula includes a plurality of ports.

Clause 20. The method of any of clauses 17-19, further comprising aligning the plurality of ports of the cutting cannula with the plurality of ports of the sample notch cannula prior to applying vacuum to the sample notch cannula.

Clause 21. The method of any of clauses 17-20, further comprising retracting the outer cannula from the sealed position to the venting position such that the outer cannula disengages the external surface of the target site once the tissue sample is severed from the target site.

Clause 22. The method of any of clause 17-21, further comprising retracting the cutting cannula from the closed position to the open position once the tissue sample is severed from the target site.

Clause 23. The method of any of clause 17-22, further comprising removing the biopsy device from the target site once the outer cannula is retracted to the venting position and the cutting cannula is retracted to the open position.

The terminology used herein is for the purpose of describing particular aspects only and is not intended to be limiting. As used herein, the singular forms “a,” “an,” and “the” are intended to include the plural forms, including “at least one,” unless the content clearly indicates otherwise. “Or” means “and/or.” As used herein, the term “and/or” includes any and all combinations of one or more of the associated listed items. It will be further understood that the terms “comprises” and/or “comprising,” or “includes” and/or “including” when used in this specification, specify the presence of stated features, regions, integers, steps, operations, elements, and/or components, but do not preclude the presence or addition of one or more other features, regions, integers, steps, operations, elements, components, and/or groups thereof. The term “or a combination thereof” means a combination including at least one of the foregoing elements.

It is noted that the terms “substantially” and “about” may be utilized herein to represent the inherent degree of uncertainty that may be attributed to any quantitative comparison, value, measurement, or other representation. These terms are also utilized herein to represent the degree by which a quantitative representation may vary from a stated reference without resulting in a change in the basic function of the subject matter at issue.

While particular embodiments have been illustrated and described herein, it should be understood that various other changes and modifications may be made without departing from the spirit and scope of the claimed subject matter. Moreover, although various aspects of the claimed subject matter have been described herein, such aspects need not be utilized in combination. It is therefore intended that the appended claims cover all such changes and modifications that are within the scope of the claimed subject matter.

1. A biopsy device, comprising:
a sample notch cannula having a proximal end and a distal end, the distal end having a sample notch configured to receive tissue from a target site;
a cutting cannula being internally located within the sample notch cannula, the cutting cannula being retractable between an open position and a closed position;
an outer cannula having a first end and a second end, the outer cannula being coaxially positioned about the sample notch cannula and retractable between a sealed position and a venting position; and
a vacuum assembly fluidly coupled to the first end of the outer cannula and the proximal end of the sample notch cannula, the vacuum assembly configured to provide a vacuum to the sample notch cannula and the outer cannula;
wherein the outer cannula applies vacuum to a surface of the target site to seal the sample notch cannula from atmosphere in the sealed position.

2. The biopsy device of claim 1, wherein the sample notch cannula comprises a plurality of ports.

3. The biopsy device of claim 1, wherein the sample notch cannula further comprises a plurality of seals.

4. The biopsy device of claim 1, wherein the cutting cannula comprises a plurality of ports.

5. The biopsy device of claim 4, wherein the plurality of ports of the cutting cannula align with the plurality of ports of the sample notch cannula when the cutting cannula is in the open position.

6. The biopsy device of claim 4, wherein the plurality of ports of the cutting cannula are misaligned with the plurality of ports of the sample notch cannula when the cutting cannula is in the closed position.

7. The biopsy device of claim 1, wherein the outer cannula disengages the surface of the target site to ventilate the sample notch cannula in the venting position.

8. The biopsy device of claim 1, wherein the outer cannula and the sample notch cannula each include a ventilation aperture, and the outer cannula is rotatable about the sample notch cannula to selectively ventilate the sample notch cannula.

9. (canceled)

10. (canceled)

11. A biopsy device, comprising:
a sample notch cannula having a proximal end, a distal end, and a plurality of ports disposed between the distal end and the proximal end, the distal end further having a sample notch configured to receive tissue from a target site;
a cutting cannula being internally located within the sample notch cannula, the cutting cannula having a plurality of ports and being retractable between an open position and a closed position;
an outer cannula having a first end and a second end, the outer cannula being coaxially positioned about the sample notch; and
a vacuum assembly fluidly coupled to the first end of the outer cannula and the sample notch cannula, the vacuum assembly configured to provide a vacuum to the sample notch cannula and the outer cannula;
wherein the plurality of ports of the cutting cannula are aligned with the plurality of ports of the sample notch cannula when the cutting cannula is in the open position and the plurality of ports of the cutting cannula are misaligned with the plurality of ports of the sample notch cannula when the cutting cannula is in the closed position.

12. The biopsy device of claim 11, wherein the plurality of ports of the cutting cannula and the plurality of ports in the sample notch cannula allow the vacuum assembly to apply a vacuum to the sample notch cannula when aligned.

13. The biopsy device of claim 11, wherein the plurality of ports of the cutting cannula and the plurality of ports in the sample notch cannula seal the sample notch cannula from atmosphere when misaligned.

14. The biopsy device of claim 11, wherein the outer cannula is further retractable between a sealed position and a venting position.

15. The biopsy device of claim 14, wherein the outer cannula applies vacuum to a surface of the target site to seal the sample notch cannula from atmosphere in the sealed position.

16. The biopsy device of claim 15, wherein the outer cannula disengages the surface of the target site to ventilate the sample notch cannula in the venting position.

17. A method of performing a biopsy comprising:
inserting a biopsy device having a sample notch cannula, a cutting cannula internally located within the sample notch cannula, an outer cannula coaxially positioned about the sample notch cannula, and a vacuum assembly fluidly coupled to the outer cannula and the sample notch cannula, into a target site;
advancing the outer cannula over the sample notch cannula from a venting position to a sealed position such that the outer cannula contacts an external surface of the target site;
applying vacuum from the vacuum assembly to the outer cannula such that the outer cannula creates a seal between the external surface of the target site and atmosphere;
applying vacuum from the vacuum assembly to the sample notch cannula to draw a tissue sample from the target site into a sample notch positioned on a distal end of the sample notch cannula; and
advancing the cutting cannula from an open position to a closed position such that the cutting cannula severs the tissue sample from the target site.

18. The method of claim 17, wherein the cutting cannula includes include a plurality of ports.

19. The method of claim 18, wherein the sample notch cannula includes a plurality of ports.

20. The method of claim 19, further comprising aligning the plurality of ports of the cutting cannula with the plurality of ports of the sample notch cannula prior to applying vacuum to the sample notch cannula.

21. The method of claim 17, further comprising retracting the outer cannula from the sealed position to the venting position such that the outer cannula disengages the external surface of the target site once the tissue sample is severed from the target site.

22. The method of claim 21, further comprising retracting the cutting cannula from the closed position to the open position once the tissue sample is severed from the target site.

23. (canceled)
